(19) United States
(12) Patent Application Publication
(10) Pub. No.: US 2013/0018366 A1
(43) Pub. Date: Jan. 17, 2013

(54) Focal Ablation Assembly

(71) Applicants: Patrick P. Wu, San Carlos, CA (US); Cesar A. Ico, San Francisco, CA (US); Richard S. Williams, Redwood City, CA (US)

(72) Inventors: Patrick P. Wu, San Carlos, CA (US); Cesar A. Ico, San Francisco, CA (US); Richard S. Williams, Redwood City, CA (US)

(73) Assignee: C2 Therapeutics, Redwood City, CA (US)

(21) Appl. No.: 13/180,443

(22) Filed: Jul. 11, 2011

(51) Int. Cl. A61B 18/02

(52) U.S. Cl. 606/21

(57) ABSTRACT

A focal ablation assembly, used with an endoscope comprising an endoscopic tube, comprises a cap and a cryogenic catheter placeable within the endoscopic tube channel. The cryogenic catheter defines a catheter lumen and has a distal end placeable at the endoscopic tube distal end. The cap is mountable to at least one of the distal ends and extends distally of both. The cap comprises a material which substantially maintains its shape during use while not causing tissue trauma. The cap defines a limited area therapeutic region, which is a chosen one of an open therapeutic region and a covered, effectively thermally transparent therapeutic region providing effectively no thermal insulation. In some examples the assembly includes a delivery catheter extending along the channel with a distal portion fluidly coupled to the cap volume, whereby refrigerant can be introduced into the cap volume and towards the therapeutic region by the delivery catheter.

### CROSS-REFERENCE TO OTHER APPLICATIONS

This application is related to the following US patent application publication: US 2010/0130970 A1 (attorney docket WILL 1002-2).

### FEDERALLY SPONSORED RESEARCH OR DEVELOPMENT

None.

### BACKGROUND OF THE INVENTION

Throughout the GI tract in the human body there are focal lesions of unwanted or unhealthy tissue that physicians desire to remove or ablate in situ. Examples of these lesions include ‘islands’ of intestinal metaplasia and dysplasia in the esophagus or ‘flat’ polyps in the colon. Removal of these tissues through techniques such as Endoscopic Mucosal Resection (EMR) may create unwanted complications such as bleeding and current ablative modalities such as Argon Plasma Coagulation (APC) and Radio Frequency Ablation (RFA) suffer from a variety of drawbacks. Furthermore, existing cryoablation technologies, which spray the cryogen directly onto the body lumen do not adequately allow control of the energy dosage.

### BRIEF SUMMARY OF THE INVENTION

An example of a focal ablation assembly is used with an endoscope comprising an endoscopic tube having proximal and distal ends and defining a channel extending between the proximal and distal ends. The focal ablation assembly comprises a cryogenic catheter and a cap. The cryogenic catheter is placeable within the channel. The cryogenic catheter defines a catheter lumen and has a distal end placeable at the distal end of the endoscopic tube. The cap is mountable to at least one of the distal end of the endoscopic tube and the distal end of the cryogenic catheter. The cap extends distally of both of the distal ends. The cap comprises a material which substantially maintains its shape during use while not causing tissue trauma. The cap defines a limited area therapeutic region. A cap volume is defined by a wall of the cap and the therapeutic region. The therapeutic region is a chosen one of an open therapeutic region and a covered, optically transparent therapeutic region providing effectively no thermal insulation. In some examples the cap is made of a flexible, soft polymer material. In some examples the cap has a distal end and the therapeutic region is at the distal end of the cap while in other examples the cap has a sidewall and the therapeutic region is at the sidewall. In some examples the focal ablation assembly includes a delivery catheter extending along the channel and has a distal portion fluidly coupled to the cap volume, whereby refrigerant can be introduced into the cap volume and towards the therapeutic region by the delivery catheter. In some examples the focal ablation assembly includes a balloon extending from the distal end of the cryogenic catheter within the therapeutic region so that the refrigerant can be introduced into the balloon within the cap volume by the delivery catheter.

Other features, aspects and advantages of the present invention can be seen on review the figures, the detailed description, and the claims which follow.

### BRIEF DESCRIPTION OF THE DRAWINGS

FIGS. 1A and 1B are overall views showing focal ablation systems made according to the invention.

FIGS. 1A-11 are directed to a first type of a focal ablation system according to a first aspect of the invention.

FIG. 2 is a simplified enlarged cross-sectional view of the distal end of one example of the system of FIG. 1A showing a cryogenic catheter passing through the channel of an endoscopic tube with a cap mounted to the distal end of the endoscopic tube, the cap having a thermally conductive therapeutic region located at the distal end of the cap.

FIG. 3 is a view similar to that of FIG. 2 of an alternative example of the system of FIG. 1A in which the therapeutic region is along a sidewall of the cap.

FIG. 4 is a view similar to that of FIG. 2 wherein the cap is mounted to the distal end of the cryogenic catheter instead of the endoscopic tube as shown in FIG. 1B.

FIGS. 5A-5C are simplified cross-sectional views of examples of three different caps in which the cross-sectional area of the therapeutic region at the distal end of each cap is different while the cross-sectional area of the endoscopic tube to which it is mounted remains the same.

FIG. 6 illustrates an example in which the therapeutic region has a convex outer surface.

FIGS. 7A, 8A and 9A are simplified end views showing caps having round, generally oval and rectangular cross-sectional shapes, respectively, each mounted to the distal end of an endoscope, each Fig. illustrating a conventional endoscope having, in this example, a camera, two lights for illumination and a working channel.

FIGS. 7B, 8B and 9B show the caps of FIGS. 7A, 8A and 9A having distal ends oriented perpendicular to the centerlines of the respective caps.

FIGS. 7C, 8C and 9C show the caps of FIGS. 7A, 8A and 9A having distal ends oriented obliquely to the centerlines of the respective caps.

FIG. 10 shows an example of a cap similar to the cap of FIG. 2 but in which the cap has an extension defining an exhaust lumen coaxial with the endoscopic tube.

FIGS. 11-15 show balloon-type of focal ablation systems according to a second aspect of the invention.

FIG. 11 shows an example of a balloon type focal ablation system in which an elastomeric balloon is mounted to the distal end of the cryogenic catheter and expands within the cap, a portion of the balloon forming a barrier to the refrigerant along the therapeutic region at the distal end of the cap.

FIGS. 12 and 12A are simplified side and end cross-sectional views of a balloon type focal ablation system in which a balloon type focal ablation assembly is used with an endoscopic tube, the balloon being mounted to the distal end of a cryogenic catheter, the cryogenic catheter passing through the channel of the endoscopic tube, the therapeutic region being along a sidewall of the balloon. FIG. 12A shows the use of reinforcing elements within the balloon to cause the balloon to have a flattened or oval cross-sectional shape to better conform to the sidewall of the body lumen.

FIGS. 13 and 13A are simplified side and end cross-sectional views of a focal ablation balloon and reinforcing elements similar to those of FIGS. 12 and 12A.

FIG. 14 illustrates another example of a balloon type focal ablation system in which the distal end of a reinforcing element extends through the delivery catheter and is secured to the distal portion of the balloon.

FIG. 15 shows a further example of a balloon type focal ablation system comprising a balloon type cap.

### DETAILED DESCRIPTION OF THE INVENTION

The following description will typically be with reference to specific structural embodiments and methods. It is to be understood that there is no intention to limit the invention to the specifically disclosed embodiments and methods but that the invention may be practiced using other features, elements, methods and embodiments. Preferred embodiments are described to illustrate the present invention, not to limit its scope, which is defined by the claims. Those of ordinary skill in the art will recognize a variety of equivalent variations on the description that follows. Like elements in various embodiments are commonly referred to with like reference numerals.

The control of three primary factors is necessary for repeatable cryoablation via evaporative cooling. These factors are evaporation temperature of the cryogen, the mass flow rate of the cryogen/surface area, and the amount of time that the cryogen is applied. The present invention directly addresses two of these factors. (1) Evaporation temperature of the cryogen is set by controlling the evaporation pressure. The evaporation pressure can be controlled by appropriately sizing the cryogenic refrigerant delivery and exhaust lumens. (2) The mass flow rate/surface area can be controlled by appropriately sizing the cryogenic refrigerant delivery lumen (to control mass flow rate) and defining a fixed treatment area either by physically defining a treatment area or by controlling distribution of the cryogen delivery onto a treatment surface.

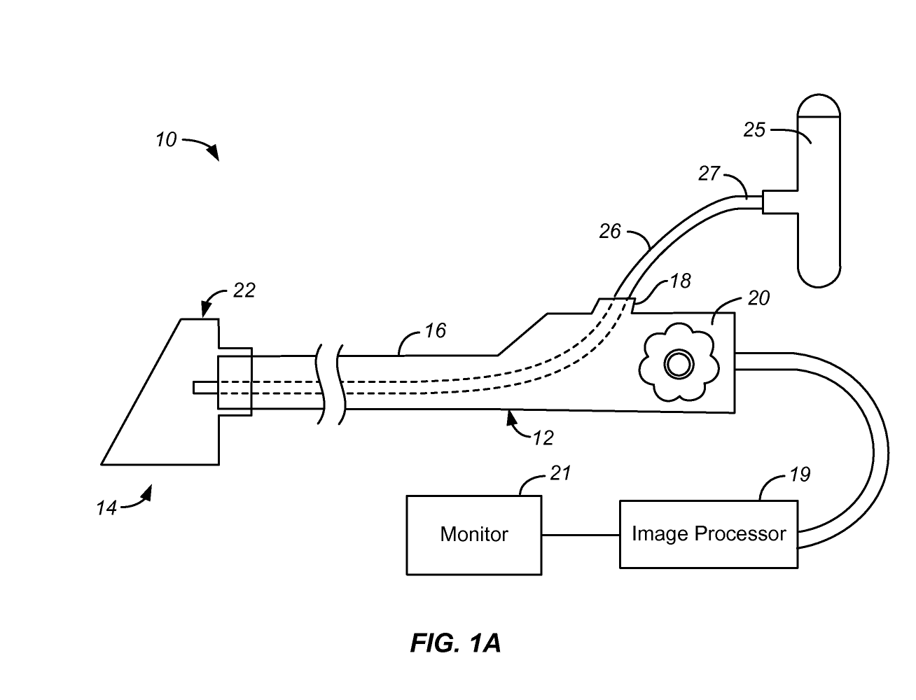

FIG. 1A is an overall view showing a generalized example of a focal ablation system 10 including broadly an endoscope 12 and a focal ablation assembly 14. Endoscope 12 includes an endoscopic tube 16 having an accessory channel port 18 at a proximal end 20 of endoscopic tube 16 and a cap 22 at the distal end 24 of endoscopic tube 16. Endoscope 12 may be a conventional endoscope such as Olympus GIF-140 or GIF-Q160Z, that connects to an image processor 19, which then displays the image on monitor 21. FIG. 1B is similar to FIG. 1A but shows cap 22 mounted to the distal end 34 of cryogenic catheter 26 instead of endoscopic tube 16.

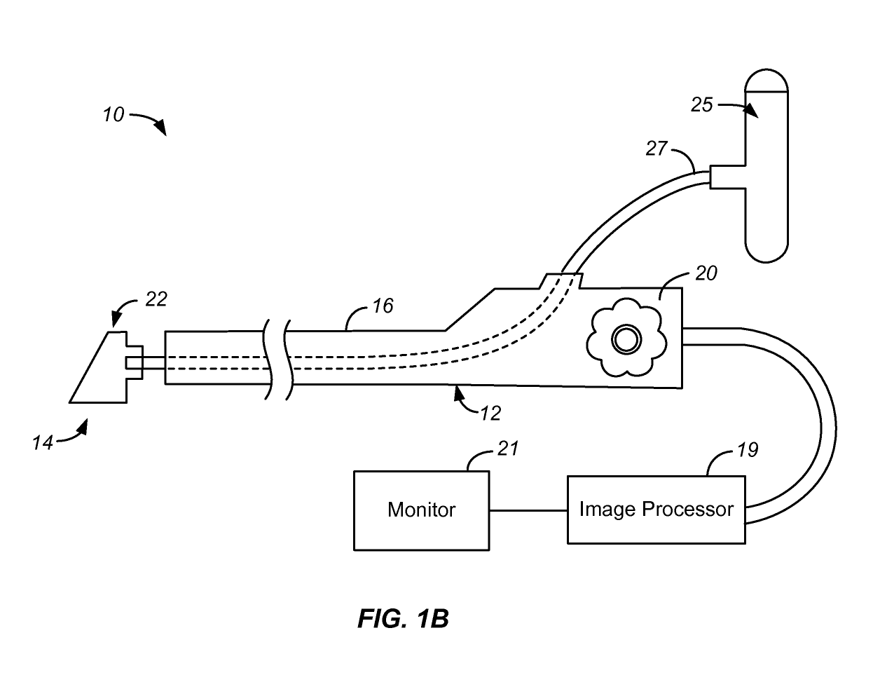

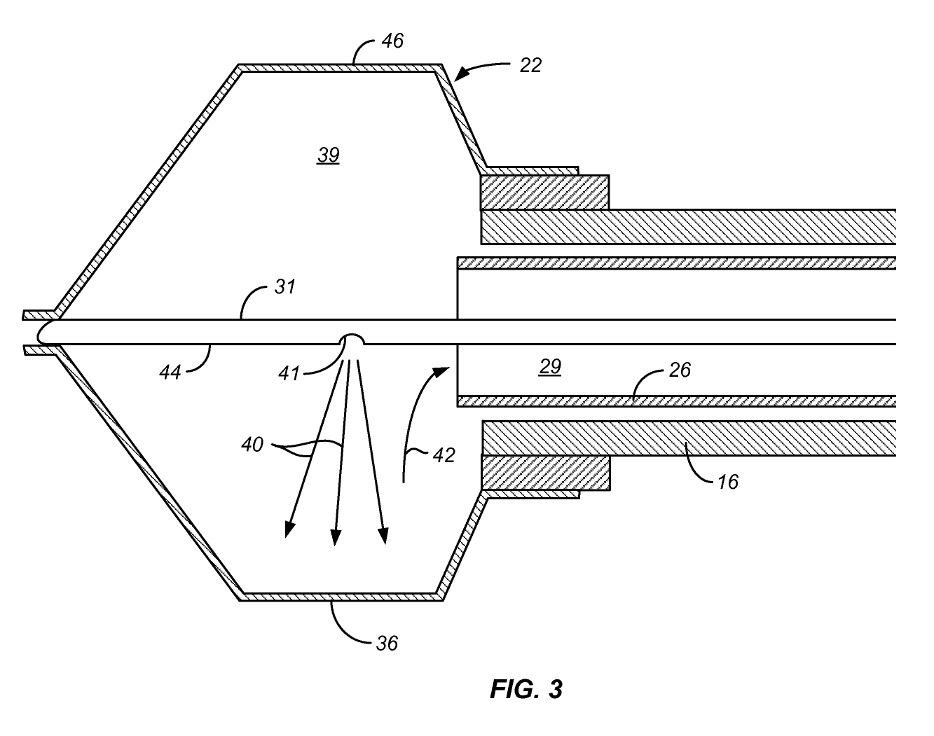

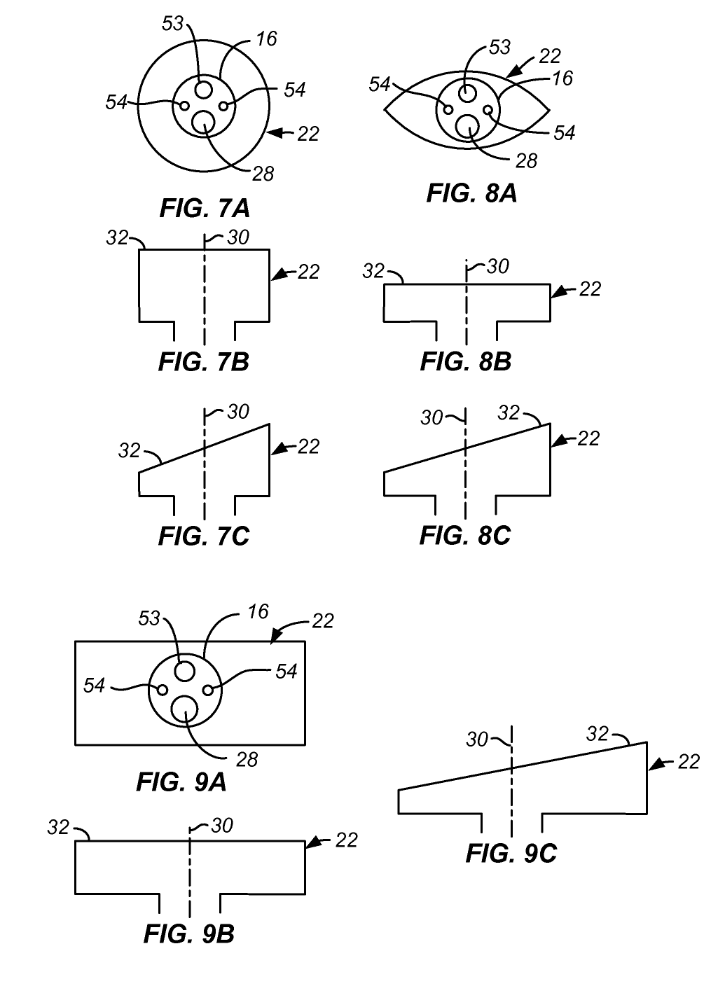

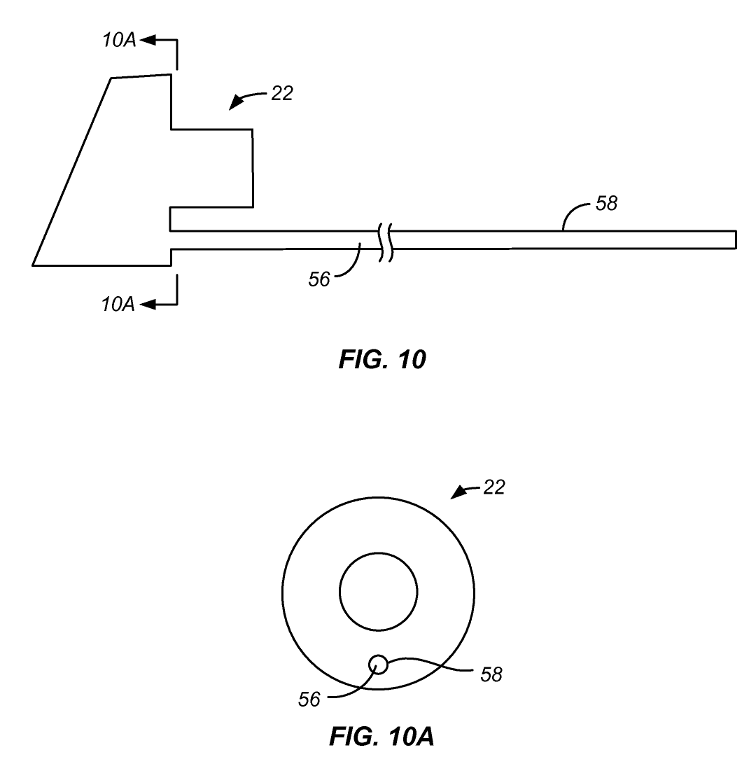

FIGS. 2-12 are directed to a first type of a focal ablation system 10 according to a first aspect of the invention. Focal ablation assembly 14, see FIGS. 1 and 2, includes a cap 22 mounted to the distal end 24 of an endoscopic tube 16 and a cryogenic catheter 26 passing through the channel 28 of endoscopic tube 16. Focal ablation assembly 14 also includes a cryoablation controller 25 at the proximal end of 27 of cryogenic catheter 26. In some examples, described below with reference to FIGS. 1B and 4, cap 22 can be mounted to the distal end 34 of cryogenic catheter 26. Cap 22 in FIG. 2 has a generally cylindrical cross-sectional shape and defines a centerline 30. Cap 22 has a distal end 32 extending distally of the distal end 24 of endoscopic tube 16 and the distal end 34 of cryogenic catheter 26. In this example cap 22 is oriented obliquely to centerline 30 to facilitate tissue apposition. Cryogenic catheter 26 defines a catheter lumen 29 through which a delivery catheter 31 passes. As is discussed in more detail below, distal end 32 of cap 22 will be placed against tissue at the target site to be treated.

Cap 22 is preferably of a clear, semi-rigid, soft, flexible material, or a combination of materials, such as a polymer material, so to substantially maintain its shape during use while not causing tissue trauma. Examples of the material for 22 include silicone, polyurethane, polyvinyl chloride, and C-Flex®, a thermoplastic elastomer, specifically styrene-ethylene-butylene modified block copolymer with silicone oil. Cap 22 may be manufactured by, for example, injection molding, casting, or thermoforming. Distal end 32 of cap 22 defines a thermally conductive therapeutic region 36 having a typical cross-sectional area of 0.5 cm² to 3.0 cm². In this example therapeutic region 36 is covered by a cover 38 of a thin transparent polymer, such as polyurethane having a thickness of typically less than 0.05 mm (0.002 inch). In this way the liquid refrigerant 40 passing through the delivery lumen of delivery catheter 31 and out through the exit opening 41 of delivery catheter 31 does not contact tissue outside the target site but rather heat is removed from the tissue at the target site as the liquid refrigerant evaporates while in contact with cover 38. Although polyurethane may not be considered to be highly thermally conductive, the thinness of cover 38 allows cover 38 to provide effectively no thermal insulation between the evaporating liquid refrigerant and the target tissue. In addition, it is preferable that cover 38 be transparent or at least translucent so that the physician can see what is happening to the tissue at the target site. The selection of the size of therapeutic region 36 is typically chosen according to the size of the treatment site or the desired mass flow rate/surface area for refrigerant 40, or both. Evaporated refrigerant 42 passes out of cap volume 39 through catheter lumen 29 between delivery catheter 31 and the inner wall of cryogenic catheter 26.

Cover 38 is stated to provide effectively no thermal insulation between the evaporating liquid refrigerant in the target tissue. In this application the phrase effectively no thermal insulation is meant to mean that tissue necrosis can occur at the target site upon the application of a cryogenically ablative liquid refrigerant, such as nitrous oxide (N₂O), to the surface of cover 38.

Cryogenic catheter 26 may be sized appropriately for introduction through, for example, 2.0 mm, 2.8 mm or 3.7 mm diameter instrument channels 28. Cryogenic catheter 26 may be constructed from materials such as PEBAX or nylon. Delivery catheter 31 may be constructed from a rigid polymer such as polyimide or a metal such as stainless steel, sufficient to withstand internal pressure approaching 1000 psig. The diameter of delivery lumen 44 defined by delivery catheter 31 is typically in the range of 0.15 mm (0.006 inch) to 0.30 mm (0.012 inch). The diameter of delivery lumen 44 can be chosen according to the desired mass flow rate/surface area for refrigerant 40 contacting therapeutic region 36.

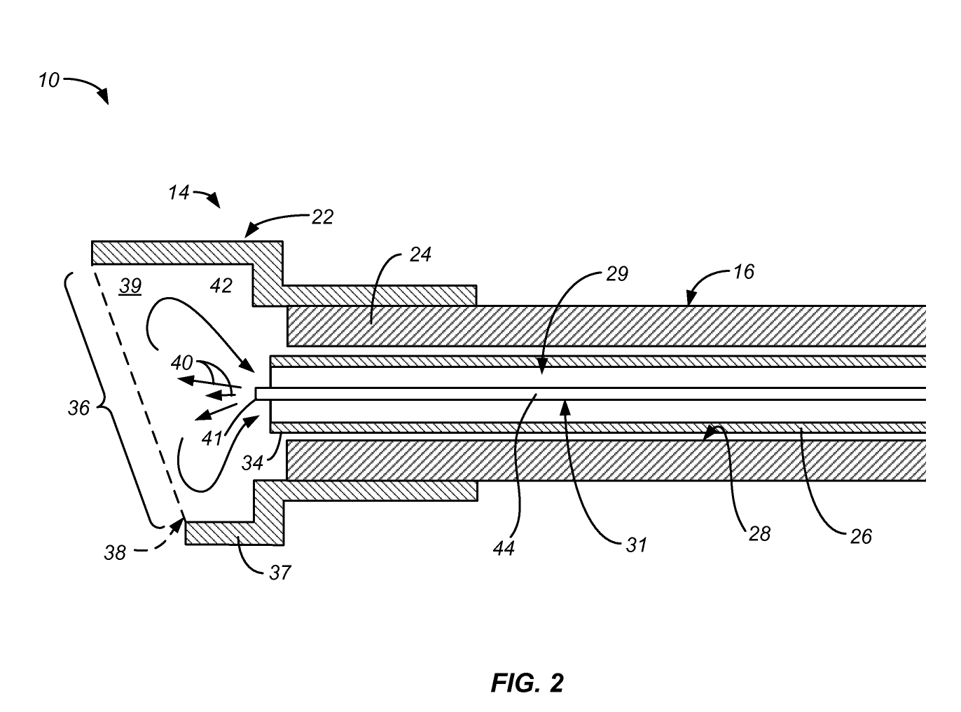

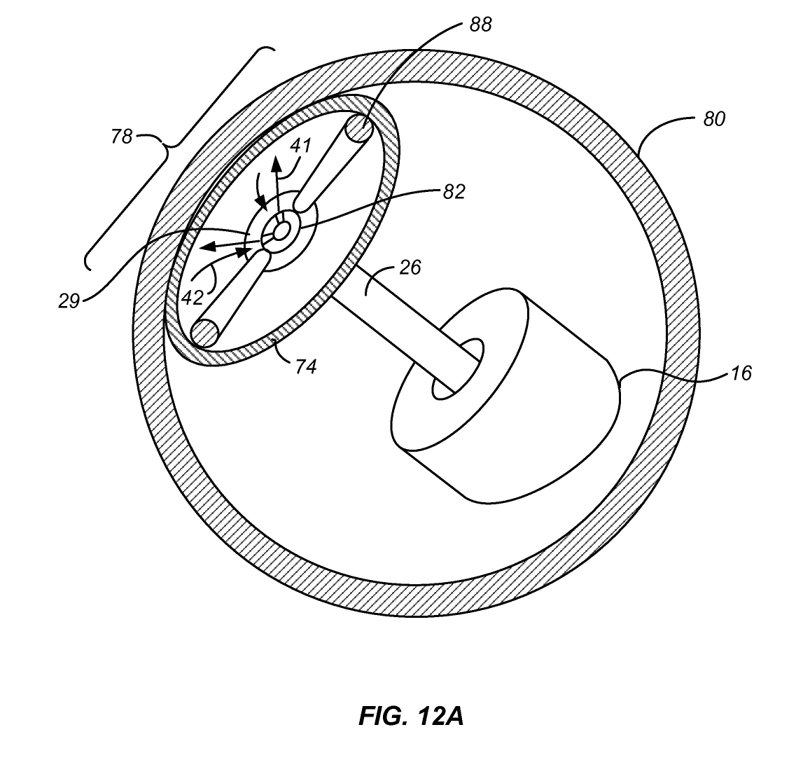

FIG. 3 is a view similar to that of FIG. 2 of an alternative example of the focal ablation system 10 of FIG. 1 in which the therapeutic region 36 is along a sidewall 35 of the cap. Delivery catheter 31 extends completely through cap volume 39 and is secured to a distal end of cap 22. Delivery catheter 31 has a laterally oriented exit opening 41 positioned opposite the therapeutic region 36 along sidewall 46 of cap 22. This feature permits liquid refrigerant 40 to be directed against sidewall 46 at therapeutic region 36 so the target tissue at the target treatment site 78 against which therapeutic region 36 is pressing, see FIG. 12, can be thermally ablated due to the low temperature of the liquid refrigerant.

FIG. 4 is a view similar to that of FIG. 2 wherein the cap 22 is mounted to the distal end 34 of the cryogenic catheter 26 instead of the endoscopic tube 16. See also FIG. 1B.

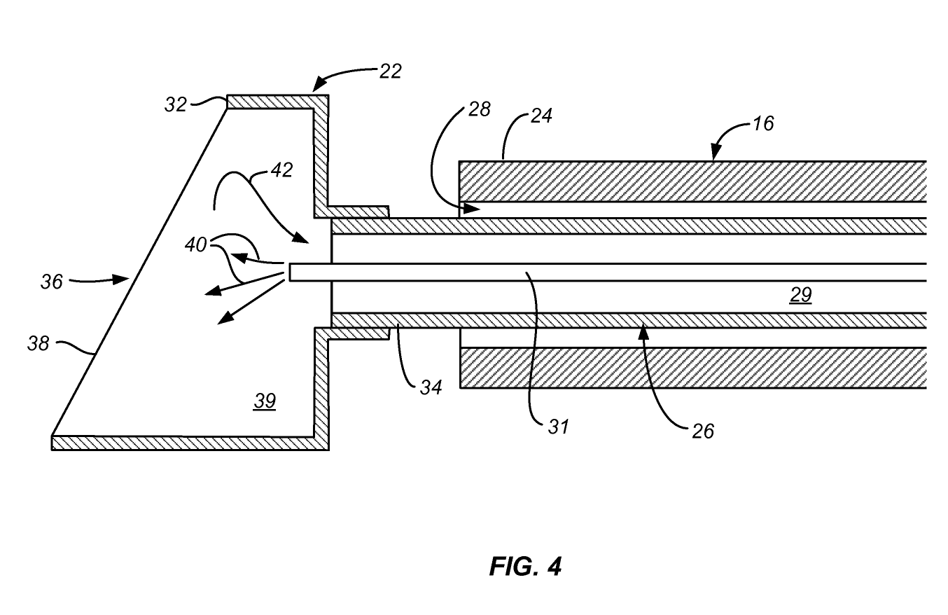

FIGS. 5A-5C are simplified cross-sectional views of examples of three different caps 22 in which the cross-sectional area 48 of the therapeutic region 36 at the distal end 32 of each cap 22 is different while the cross-sectional area 50 of the endoscopic tube 16 to which it is mounted remains the same. This concept is used to allow the physician to choose the appropriately sized cap 22 according to the size of the target site, or the mass flow rate/surface area of refrigerant 40, or both.

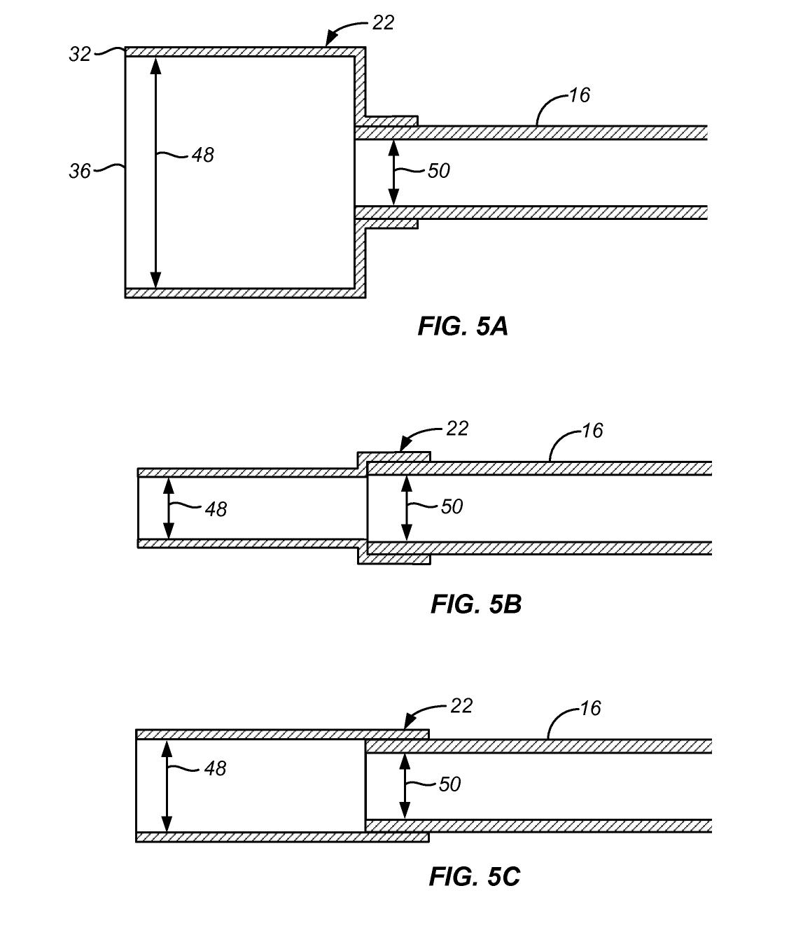

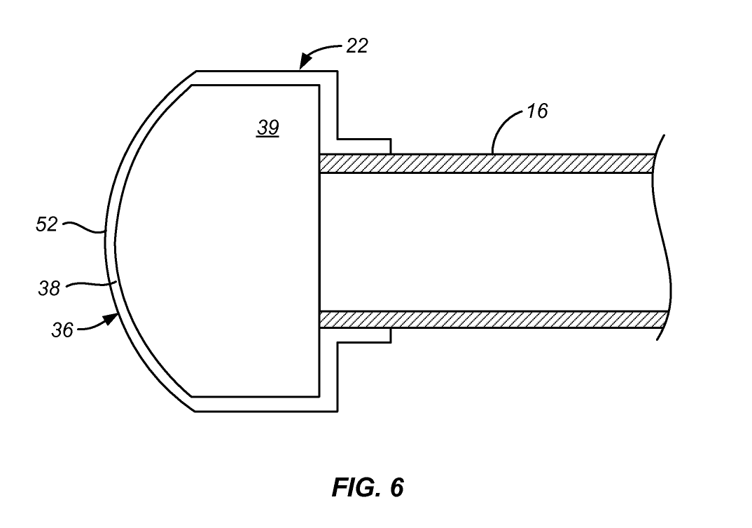

FIG. 6 illustrates an example in which the therapeutic region 36 has a convex outer surface 52. This configuration may be useful to improve the amount of contact between the therapeutic surface of the cap and the target tissue. FIGS. 7A, 8A and 9A are simplified end views showing caps 22 having round, generally oval and rectangular cross-sectional shapes, respectively. Each cap 22 is mounted to the distal end 24 of an endoscopic tube 16. Each figure illustrates a conventional endoscope 12 having, in this example, a camera 53, two lights 54 for illumination and a working channel 28.

FIGS. 7B, 8B and 9B show the caps 22 of FIGS. 7A, 8A and 9A having distal ends 32 oriented perpendicular to the centerlines 30 of the respective caps. FIGS. 7C, 8C and 9C show the caps 22 of FIGS. 7A, 8A and 9A having distal ends 32 oriented obliquely to the centerlines 30 of the respective caps.

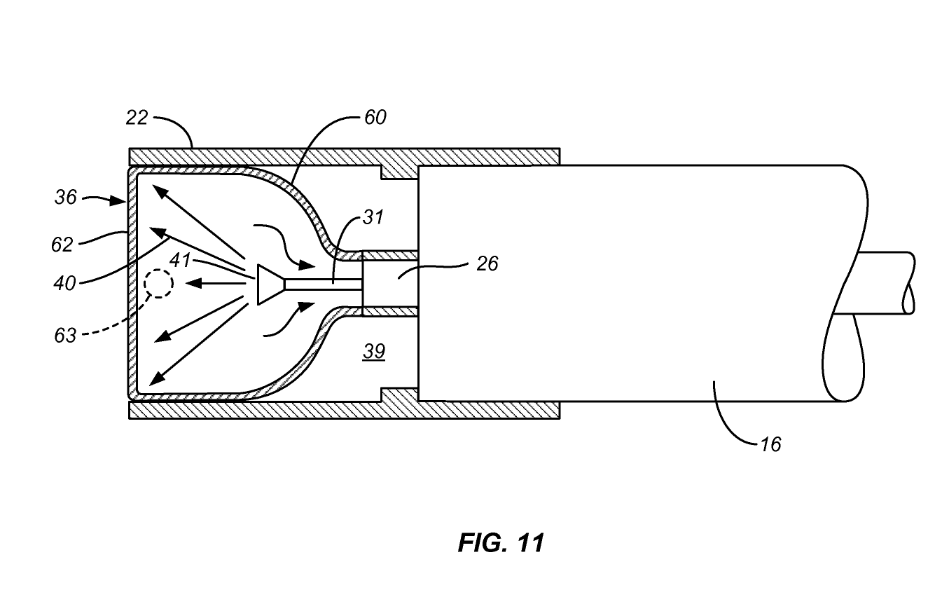

FIG. 10 shows an example of cap 22 similar to the cap 22 of FIG. 2 but in which an exhaust lumen 56 is defined by a coaxial extension 58 of cap 22. Extension 58 will lie generally parallel to the endoscopic tube 16. This permits and a larger cross-sectional area for the exhaust lumen than would be typically available if the exhaust lumen was defined by an accessory channel of endoscopic tube 16. Exhaust lumen 56 is typically in the range of 2-5 mm in diameter. The cross-sectional areas provided by catheter lumen 29 when used as exhaust lumens in the above examples are typically of a similar size.

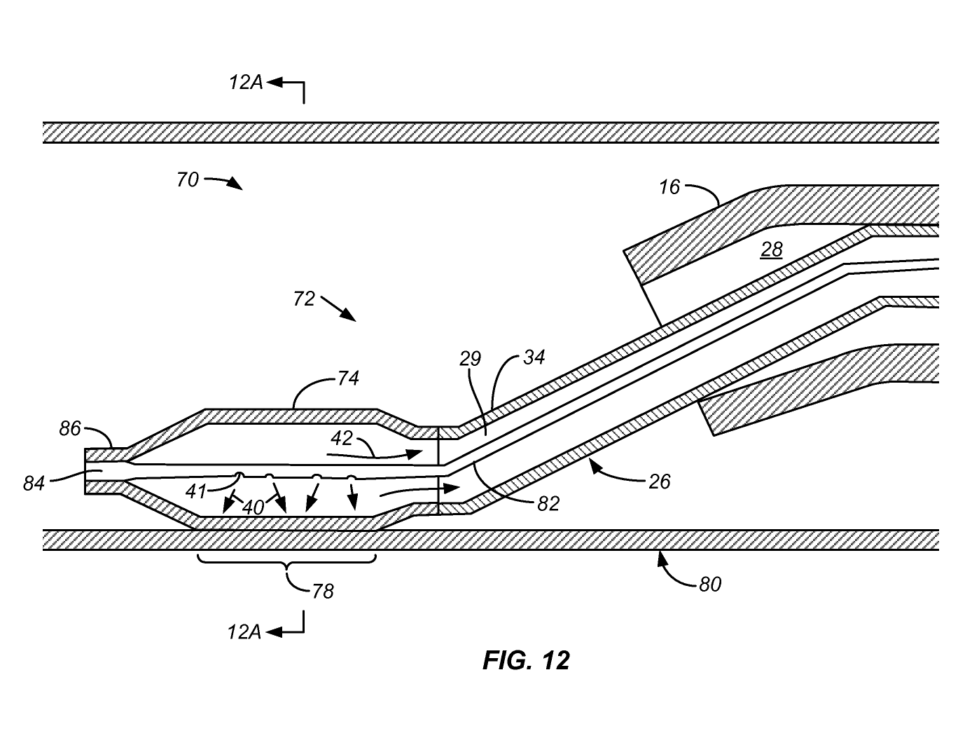

FIG. 11 shows another example in which an elastomeric balloon 60 is mounted to the distal end 34 of the cryogenic catheter 26. Balloon 60 expands within the cap volume 39 of cap 22 with a portion 62 of the balloon forming the cover 38 along the therapeutic region 36 at the distal end 32 of the cap. A vent hole 63 is formed in the sidewall 35 of cap 22 to facilitate the expansion of balloon 60.

In use, the physician will typically select a cap 22 having the appropriate size and shape for the particular target treatment site 78. The size of therapeutic region 36 may also be chosen according to the desired mass flow rate/surface area for refrigerant 40. Assuming the focal ablation system 10 of FIG. 2 is being used, cap 22 can be installed on the distal end 24 of endoscopic tube 16. Cryogenic catheter 26, typically with delivery catheter 31 therein, can be placed through accessory port 18 of the endoscope 12 and passes through channel 28 of the endoscopic tube 16 until the distal end 34 of cryogenic catheter 26 is at cap volume 39. Note that the installation of cap 16 could occur after positioning cryogenic catheter 26 within endoscope 12. The distal portion of focal ablation system 10 is placed in the patient so that region 36 is properly positioned at the target treatment site 78. The refrigerant from cryoablation controller 25 is then directed through delivery catheter 31 and against cover 38 at to the target treatment site 78. The evaporation of the refrigerant on cover 38 lowers the temperature of the tissue at the target treatment site enough to cause necrosis of the tissue. The evaporated refrigerant 42 passes out of cap volume 39 through catheter lumen 29.

FIGS. 12-15 show balloon type of focal ablation systems, somewhat different from the focal ablation system of FIGS. 1-11, according to a second aspect of the invention.

FIGS. 12 and 12A are simplified side and end cross-sectional views of a balloon type focal ablation system 70 in which a balloon type focal ablation assembly 72 is used with an endoscopic tube 16 of an endoscope 12. A balloon 74 is mounted to the distal end 34 of cryogenic catheter 26. The cryogenic catheter 26 extends through the channel 28 of endoscopic tube 16. The therapeutic region 36 is along a sidewall 76 of balloon 74. In this example cryogenic catheter 26 has a dogleg shape distal portion to facilitate placement of therapeutic region 36 along sidewall 76 and against a target treatment site 78 of the body structure 80. Delivery catheter 82 passes through lumen 29 with its distal end 84 secured to the distal end 86 of balloon 74. Delivery catheter 82 has, in this example, a number of laterally directed exit openings 41 acting as delivery ports to direct liquid refrigerant 40 into the interior 85 of balloon 74 and against sidewall 76 of balloon 74 at target site 78. The size, number and positions of openings 41 can be chosen according to the size of the target treatment site 78 and desired mass flow rate/surface area for refrigerant 40. FIG. 12A shows the use of reinforcing elements 88, such as nitinol support wires, within the balloon 74 to cause the balloon to have a flattened or oval cross-sectional shape to better conform to the shape of the body structure 80. The use of reinforcing elements 88 also helps to maintain balloon 74 in direct contact with the body structure during the procedure.

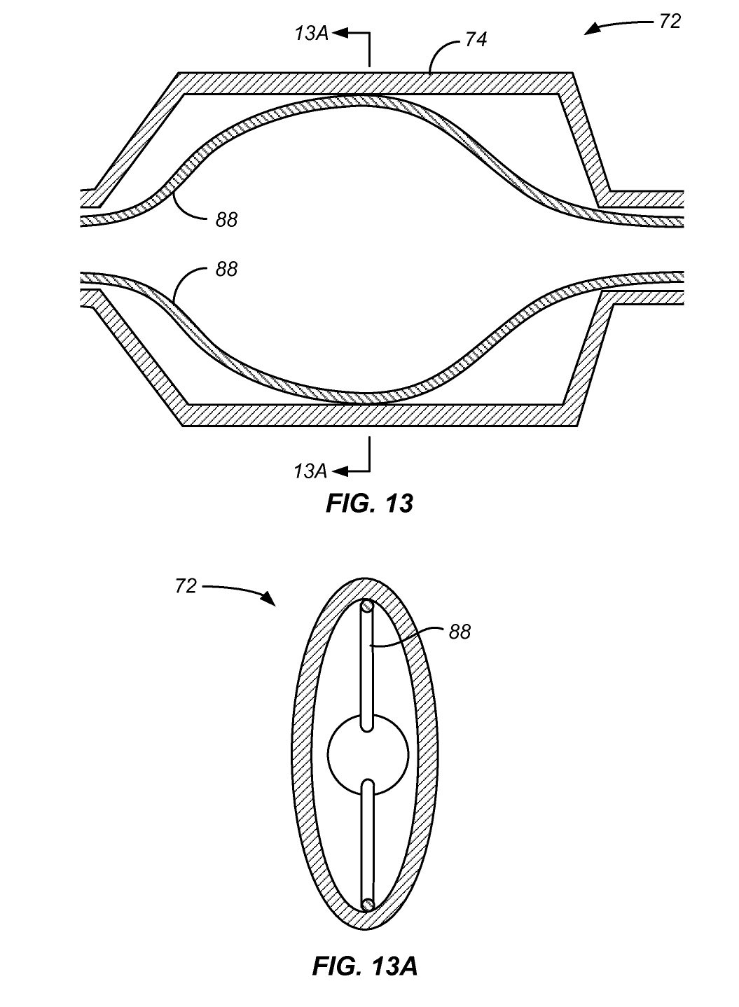

FIGS. 13 and 13A are simplified side and end cross-sectional views of a focal ablation balloon 74 and reinforcing elements 88 similar to those of FIGS. 12 and 12A.

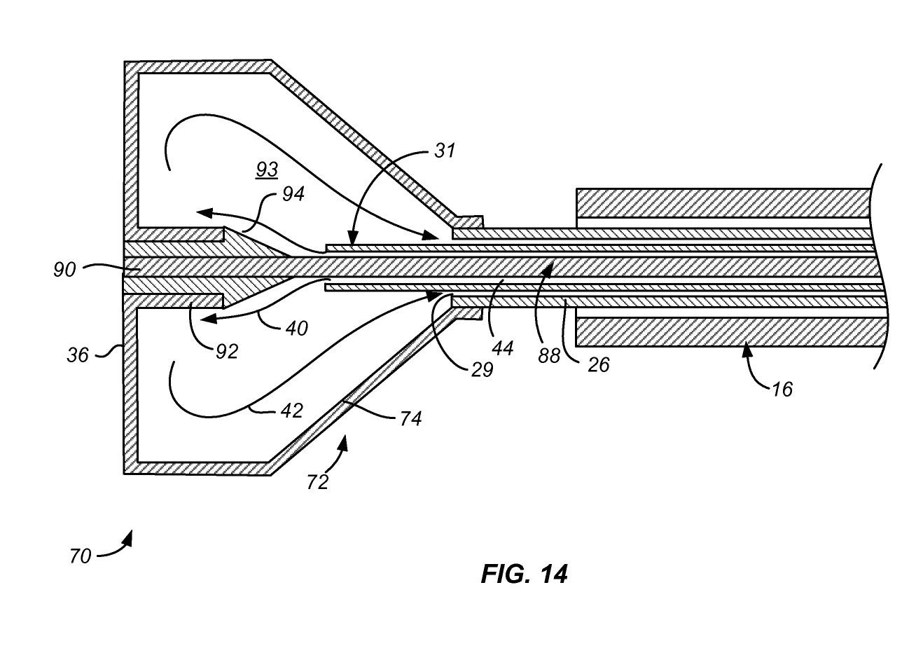

FIG. 14 illustrates another example of a balloon type focal ablation system 70 in which the distal end of the reinforcing element 88 extends through the delivery catheter 31. The distal end 90 of reinforcing element 88 is secured to the central portion of the working region 36 of balloon 74 in the following manner. Balloon 74 has a stem portion 92 at the center of therapeutic, working region 36 extending inwardly into the volume 93 defined by the balloon. A thermally conductive filler material 94 is used to secure distal end 90 of reinforcing element 88 to stem portion 92. As indicated by liquid refrigerant arrows 40, liquid refrigerant is directed toward the center of working region 36 surrounding stem portion 92.

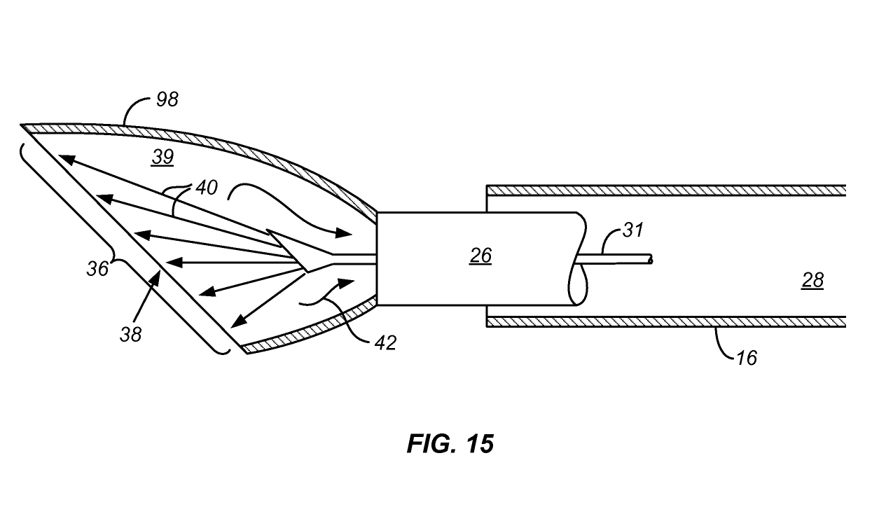

FIG. 15 shows an example of a balloon type focal ablation system comprising a balloon type cap 98, which is not generally rigid as caps 22 in the examples above FIGS. 1-11. Rather, cap 98 is typically made of one or more materials similar to those used with balloon 74 and includes reinforcing elements 88, not shown in FIG. 15, which may be made of, for example, a metal, such as nitinol, or of the same material as the rest of cap 98. Cap 98 also includes a cover 38, typically made of a thin film of silicone, polyurethane, or PET. As with the balloon type focal ablation systems 70 of FIGS. 12-14, the full expansion of cap 98 is typically the result of reinforcing elements 88 and the internal pressure created by the vaporization of refrigerant 40 and expansion due to the creation of exhaust gas 42.

In the use of the focal ablation system 70 of FIGS. 12-13A, the physician will typically select a focal ablation assembly 72 including a balloon 74 having the appropriate size and shape for the particular target treatment site 78. Cryogenic catheter 26, typically with delivery catheter 82 therein and the balloon 74 at the distal end 34, can be placed through accessory port 18 of the endoscope 12 and pass through channel 28 of the endoscopic tube 16 until sidewall 76 of balloon 74 is adjacent to therapeutic region 36 of body structure 80. The refrigerant 40 from cryoablation controller 25 is then directed through delivery catheter 82 and against balloon sidewall 76 at to the target treatment site 78. The evaporation of the refrigerant on sidewall 76 of balloon 74 lowers the temperature of the tissue at the target treatment site 78 enough to cause necrosis of the target tissue. The evaporated refrigerant 42 passes out of the interior of balloon 74 through catheter lumen 29. The use of the examples of FIGS. 14 and 15 are carried out in similar manners with the working region 36 positioned at target treatment site 78

The above descriptions may have used terms such as above, below, top, bottom, over, under, et cetera. These terms may be used in the description and claims to aid understanding of the invention and not used in a limiting sense.

While the present invention is disclosed by reference to the preferred embodiments and examples detailed above, it is to be understood that these examples are intended in an illustrative rather than in a limiting sense. It is contemplated that modifications and combinations will occur to those skilled in the art, which modifications and combinations will be within the spirit of the invention and the scope of the following claims. For example, cryogenic catheter 26 may also contain features related to measuring the performance of the system for either safety or efficacy reasons. Examples of these features include a pressure sensing lumen for monitoring pressure in the volumes 39, 93, and a temperature sensing device (e.g. thermistor or thermocouple) for monitoring temperature in the cap 22, especially at working region 36.

Any and all patents, patent applications and printed publications referred to above are incorporated by reference.

What is claimed is:

1. A focal ablation assembly, for use with an endoscope comprising an endoscopic tube having proximal and distal ends and defining a channel extending between the proximal and distal ends, the focal ablation assembly comprising:
a cryogenic catheter placeable within the channel, the cryogenic catheter defining a catheter lumen and having a distal end placeable at the distal end of the endoscopic tube;
a cap mountable to at least one of the distal end of the endoscopic tube and the distal end of the cryogenic catheter, the cap extending distally of both of the distal ends;
the cap comprising a material which substantially maintains its shape during use while not causing tissue trauma;
the cap defining a limited area therapeutic region;
the cap defining a cap volume defined by a wall of the cap and the therapeutic region; and
the therapeutic region being a chosen one of:
an open therapeutic region; and
a covered, optically transparent therapeutic region providing effectively no thermal insulation.

2. The assembly according to claim 1, wherein the cap is made of a flexible, soft polymer material.

3. The assembly according to claim 1, wherein the cap is made of at least one of polyvinyl chloride and polyurethane.

4. The assembly according to claim 1, wherein the cap has a distal end and the therapeutic region is at the distal end of the cap.

5. The assembly according to claim 1, wherein the cap has a sidewall and the therapeutic region is at the sidewall.

6. The assembly according to claim 1, wherein the therapeutic region is the covered therapeutic region, the covered therapeutic region having a flat shape.

7. The assembly according to claim 1, wherein the therapeutic region is the covered therapeutic region, the covered therapeutic region having a convex shape.

8. The assembly according to claim 1, wherein the therapeutic region is the covered therapeutic region, the covered therapeutic region being covered by a flexible, tissue-conformable material.

9. The assembly according to claim 1, wherein the therapeutic region is the covered therapeutic region, the covered therapeutic region being covered by an elastomeric material.

10. The assembly according to claim 1, wherein the endoscopic tube has a centerline and the therapeutic region is oriented oblique to the centerline.

11. The assembly according to claim 1, wherein the therapeutic region is generally elliptical.

12. The assembly according to claim 1, wherein the therapeutic region has a cross-sectional area and the endoscopic tube has a cross-sectional area, the cross-sectional area of the therapeutic region being greater than the cross-sectional area of the endoscopic tube.

13. The assembly according to claim 1, further comprising a delivery catheter extending along the channel and having a distal portion fluidly coupled to the cap volume, whereby refrigerant can be introduced into the cap volume and towards the therapeutic region by the delivery catheter.

14. The assembly according to claim 13, wherein the delivery catheter extends through the cryogenic catheter.

15. The assembly according to claim 14, further comprising:
a balloon extending from the distal end of the cryogenic catheter within the therapeutic region so that the refrigerant can be introduced into the balloon within the cap volume by the delivery catheter; and
an exhaust lumen defined by the catheter lumen.

16. The assembly according to claim 13, wherein the distal portion of the delivery catheter is positioned with an exit opening positioned within the cap volume.

17. The assembly according to claim 13, further comprising an exhaust lumen defined by at least one of (1) the channel of the endoscopic tube, and (2) the catheter lumen.

18. The assembly according to claim 13, wherein the distal portion of the refrigerant delivery catheter has a single exit opening for the discharge of the refrigerant towards the therapeutic region.

19. A focal ablation system comprising:
an endoscope comprising an endoscopic tube having proximal and distal ends and defining a channel extending between the proximal and distal ends;
a focal ablation assembly comprising a cryogenic catheter and a cap;
the cryogenic catheter located within the channel, the cryogenic catheter defining a catheter lumen and having a distal end at the distal end of the endoscopic tube;
the cap mounted to at least one of the distal end of the endoscopic tube and the distal end of the catheter, the cap extending distally of both of the distal ends;
the cap defining a fixed-area therapeutic region;
the cap defining a volume defined by a wall of the cap and the therapeutic region; and
the therapeutic region being a chosen one of:
an open therapeutic region; and
a optically thermally transparent therapeutic region providing effectively no thermal insulation.

20. The system according to claim 19, further comprising a delivery catheter extending along the channel and having a distal portion fluidly coupled to the cap volume, whereby refrigerant can be introduced into the cap volume and towards the therapeutic region by the delivery catheter.

21. The system according to claim 20, further comprising an exhaust lumen defined by at least one of (1) the channel of the endoscopic tube, and (2) the catheter lumen.
